(19) United States
(12) Patent Application Publication
(10) Pub. No.: US 2014/0205741 A1
(43) Pub. Date: Jul. 24, 2014

(54) PROCESS FOR MAKING PERSONAL CARE ARTICLES

(71) Applicant: The Procter & Gamble Company, Cincinnati, OH (US)

(72) Inventors: Clifford Theodore PAPSDORF, Loveland, OH (US); Brian Charles CRENSHAW, Mason, OH (US)

(73) Assignee: The Procter & Gamble Company, Cincinnati, OH (US)

(21) Appl. No.: 14/163,007

(22) Filed: Jan. 24, 2014

Related U.S. Application Data
(60) Provisional application No. 61/756,106, filed on Jan. 24, 2013.

(51) Int. Cl. A61K 8/02; A61Q 9/04

(52) U.S. Cl. CPC A61K 8/0233; A61Q 9/04
USPC 427/2.31

(57) ABSTRACT

A process for coating a personal care composition onto at least one discrete treatment area of a substrate having a first and second surface wherein the personal care composition has a yield point of from 10 Pa to 2000 Pa measured via a stress controlled amplitude sweep at a frequency of 1 Hz and a temperature of 25° C. comprising applying the personal care composition to a portion of the first surface of the substrate; providing a support member with a support surface wherein the second surface of the substrate contacts the support surface of the support member, the support surface having at least one cavity corresponding to the treatment area; providing a wiping member in contact with the first surface of the substrate; deforming the substrate with the applied personal care composition on its first surface into the cavity such that the resulting stress created due to the deformation of the substrate into cavity is less than the elastic limit of the substrate; providing relative motion between the support member and the wiping member such that excess personal care composition is removed and the substrate is coated with a discrete area of personal care composition substantially corresponding to the cavity; wherein the substrate is not permanently deformed; and removing the substrate from the support member.

### FIELD OF THE INVENTION

The present invention relates to processes for the coating of compositions onto substrates, more specifically to the production of personal care articles.

### BACKGROUND OF THE INVENTION

Personal care compositions used to provide benefits to mammalian keratinous tissue are known, for example by moisturizing/hydrating, colouring, nourishing or otherwise improving the visible and/or tactile condition of keratinous tissue. In particular, depilatory compositions may comprise reducing agents to degrade keratin in the hair and thus weaken the hair strands to enable the removal of unwanted hair. Personal care compositions may take the form of creams, gels, lotions and the like which may be applied to the keratinous tissue in a variety of ways, such as with a spatula or by hand. This can be a messy and awkward procedure for the user of the personal care composition, for example by resulting in personal care composition coming into contact with the hands. By disposing the personal care composition on a material one may overcome or mitigate such disadvantages. Material-based personal care products, in particular depilatories, are known from JP63073910A, US2006002878, JP6135826A, JP11012123A and JP62230711A. Processes for coating compositions onto substrates are known from U.S. Pat. No. 3,218,776, U.S. Pat. No. 2,513,852 and U.S. Pat. No. 4,375,146.

While addressing some of the usage problems of creams and lotions by removing some of the messiness created, known methods to produce substrate-based personal care articles and known processes for coating compositions onto substrates do not address the problem of achieving a shaped area of personal care composition upon the substrate, to enable easy handling of the personal care article. Furthermore, they do not address the selective application of the personal care composition to part of a surface, or in other words to enable the area covered by personal care composition to fit to certain parts of the body. This requires the substrate to not permanently deform during the coating process otherwise the final article will not be fit for purpose. There exists a need, therefore, for a process that readily facilitates the production of a personal care article having a shaped area of composition disposed upon a substrate. Furthermore, when the personal care composition is a depilatory, there is a need to provide a depilatory composition which is of the required thickness to coat the hair. This problem is not addressed by the coating processes of the prior art.

### SUMMARY OF THE INVENTION

The present invention relates to a process for coating a personal care composition onto at least one discrete treatment area of a substrate having a first and second surface wherein the personal care composition has a yield point of from 10 Pa to 2000 Pa measured via a stress controlled amplitude sweep at a frequency of 1 Hz and a temperature of 25° C. comprising the steps of:
a. applying the personal care composition to a portion of the first surface of the substrate
b. providing a support member with a support surface wherein the second surface of the substrate contacts the support surface of the support member, the support surface having at least one cavity corresponding to the treatment area;
c. providing a wiping member in contact with the first surface of the substrate;
d. deforming the substrate with the applied personal care composition on its first surface into the cavity such that the resulting stress created due to the deformation of the substrate into cavity is less than the elastic limit of the substrate;
e. providing relative motion between the support member and the wiping member such that excess personal care composition is removed and the substrate is coated with a discrete area of personal care composition substantially corresponding to the cavity; wherein the substrate is not permanently deformed; and
f. removing the substrate from the support member.

Preferably steps a-f are performed in the order listed above.

### BRIEF DESCRIPTION OF THE DRAWINGS

FIG. 1 illustrates a front perspective view of a process for coating a discrete pattern of personal care composition;

FIG. 2 illustrates a side cut away view of a process for coating a discrete pattern of personal care composition;

FIG. 3 illustrates an exploded perspective view of a support member and a vacuum manifold;

FIG. 4 illustrates a fragmented side cut away view showing a discrete area of personal care composition coated after distribution and removal by a wiping member;

FIG. 5 illustrates a combination of a coated substrate with a second substrate resulting in a combined coated composite web;

FIG. 6 illustrates a fragmented side cut away view showing an alternative embodiment with a discrete area of personal care composition coated after distribution and removal by a wiping member without negative pressure supplied to the cavities on the support member;

FIG. 7 illustrates a front perspective view of an alternative embodiment with a process for coating a discrete pattern of personal care composition wherein the support surface is flat;

FIG. 8 illustrates a side cut away view of an alternative embodiment with a process for coating a discrete pattern of personal care composition wherein the support surface is flat;

FIG. 9 illustrates a side cut away view of another alternative embodiment with a process for coating a discrete patterns of personal care composition wherein the support surface is flat without negative pressure supplied to the cavities on the support member;

FIG. 10 illustrates a partially exploded perspective view of a personal care article showing different shapes for discrete area of personal care composition, cut area for substrate, and cut area for second substrate;

FIG. 11 illustrates a front perspective view of a process for making a personal care article by kiss cutting a coated substrate and second substrate.

### DETAILED DESCRIPTION OF THE INVENTION

The process of the present invention comprises coating a personal care composition onto a treatment area of a plastic substrate. Preferably the personal care composition is a depilatory composition, more preferably an aqueous depilatory composition.

The personal care composition must have a particular rheology in order to remain deposited on the substrate and deform during use. The yield point describes the resistance of the personal care composition to deformation under environmental stress. If the yield point is too high, then the composition may not deform sufficiently. When the composition is a depilatory composition this will result in hair fibres unable to enter the depilatory composition effectively upon application, resulting in less desirable depilatory effectiveness. If the yield point is too low, however, then the composition may flow during storage, transport or use and is not cleanly removed from the skin upon removal of the article, thus requiring the inconvenience of additional wiping and where the composition is a depilatory, risking irritation to the user. Accordingly, the phase angle of the composition preferably has a yield point from 10 Pa to 2000 Pa, more preferably from 30 Pa to 1200 Pa, even more preferably from 45 Pa to 500 Pa and even more preferably still from 60 Pa to 300 Pa, when measured via a stress controlled amplitude sweep at a frequency of 1 Hz and a temperature of 25° C. The yield point described is defined as the 5% decrease in magnitude of the elastic modulus G′ linear viscoelastic plateau value as measured on a TA1000 Rheometer, available from TA Instruments of New Castle, Del., USA. The skilled person will understand that the rheological properties of the composition may be altered by changing the concentration or identity of the thickening system and the water content of the composition.

The substrate preferably comprises at least one water impermeable plastic and is compatible with the personal care composition. Using a water impermeable substrate prevents water loss from the composition while the composition is in contact with the tissue and thus prevents the composition from drying out. Water loss from the composition lowers the water concentration, thus increasing the concentration of active ingredients. This could result in irritation to the skin, which applicants wish to avoid. Examples of useful water impermeable plastics include but are not limited to polypropylene (PP); polyethylene (PE, including HDPE and LLDPE); polyethylene terephthalate (PET); polyvinylchloride (PVC); polyamide (PA); polycarbonate; polyurethane; cellulose acetate; polychloropene; polysulfone; polytetrafluoroethylene (PTFE); polyvinyl acetate (PVA); polystyrene; polyphenylene oxide (PPO); acrylonitrile butadiene styrene (ABS); acrylic; acrylonitrile styrene acrylate (ASA); ethylene vinyl alcohol (EVA); natural rubber, latex, nylon, nitrile, silicone and thermo plastic elastomers (TPE). The substrate may comprise a single polymer or mixtures of polymers or copolymers. Preferably the substrate comprises or consists of a polyolefin, preferably a polyethylene and even more preferably a high density polyethylene.

Preferably there is no layer of water permeable material between the composition and the plastic substrate in order to reduce water loss for the reasons detailed above. In a preferred embodiment, the water impermeable plastic material forms a water impermeable layer.

The substrate preferably possesses a rigidity in the range of from 5.00 g/cm to 0.08 g/cm, more preferably from 3.00 g/cm to 0.08 g/cm, even more preferably from 1.80 g/cm to 0.10 g/cm, even more preferably still from 0.80 g/cm to 0.15 g/cm and yet more preferably from 0.60 g/cm to 0.25 g/cm. This rigidity of the substrate ensures that desirable handleability and conformability attributes of an article are achieved as well as helping to ensure that the substrate does not permanently deform during the coating process. In particular, the preferred rigidity helps to prevent the article collapsing under gravity or folding, which is especially undesirable if different areas of composition are able to readily come into contact with each other, while maintaining the capability for the substrate to conform to the surface to which it is applied without folding or crinkling, in order to further improve depilatory efficiency. Accordingly, the substrate is readily conformable to the skin and if applicable, any unwanted hair, without permanently deforming during use or during production, as this may also result in problems for the user during application. In a preferred embodiment, the rigidity is substantially constant and does not change during the lifetime of a product.

Rigidity can be readily measured using the American Standard Test Method (ASTM) D2923-06, method B (i.e. using a powder to reduce the effect of static electricity) on a Handle-O-Meter, model #211-300, available from Thwing-Albert Instrument Co. of Philadelphia, Pa. The rigidity is expressed as grams per centimetre of sample width. Samples were prepared as 10.16 cm (4 inch) by 10.16 cm (4 inch) test specimens with edges parallel to the machine direction and transverse direction for substrates with directionality. Three rigidity measurements were determined on the same side of fresh test specimens orientated in the same substrate direction. A further three rigidity measurements were taken on the same side of fresh test specimens oriented at 90° to the first orientation. These six measurements were repeated on the opposite side to the first six measurements, on fresh test samples. The 12 rigidity measurements were then averaged and reported to 0.01 g/cm.

The rigidity of a substrate is a function of substrate thickness and inherent modulus of elasticity. Different materials have different moduli of elasticity. Based upon the material or materials that the substrate comprises, a substrate thickness should be selected that enables the desired rigidity of the substrate to be achieved.

The substrate preferably has a thickness of from 80 μm to 12 μm, more preferably from 50 μm to 15 μm, even more preferably from 40 μm to 16 μm and even more preferably still from 30 μm to 17 μm.

Non-limiting examples of substrate material and thickness combinations are:

| Substrate Material | Thickness [microns] | Rigidity [g/cm] |
|---|---|---|
| HDPE | 13 | 0.13 |
| HDPE | 18 | 0.33 |
| HDPE | 36 | 1.05 |
| LLDPE | 23 | 0.23 |
| PP | 18 | 0.46 |

[HDPE is a mixture of LBI 85% M6030 and Exxon Mobil 15% LD2001 manufactured on a Merritt-Davis casting line]
[LLDPE is Exxon Mobil 15% LD2001 manufactured on a Merritt-Davis casting line]
[PP is Basell PH835 manufactured on a Merritt-Davis casting line]

The substrate comprises a first and second surface. The substrate may comprise a textured or, alternatively, micro-structured surface on at least a portion of one surface. Surface texturing or micro-structuring increases the effective surface area of the substrate and thus improves adherence of the composition to said substrate, facilitating an easy removal of the article by peeling it off the skin, or increases the grip of the surface, thus improving handleability. The textured structures may comprise dimples; lines or curvilinear embossments. A textured surface may be formed on the substrate by any appropriate technique, including embossment calendars and casting.

The substrate may be manufactured by any suitable method, including casting, injection moulding, co-injection moulding, over moulding, in-mold assembly, compression moulding, blow moulding, casting thermo or vacuum forming.

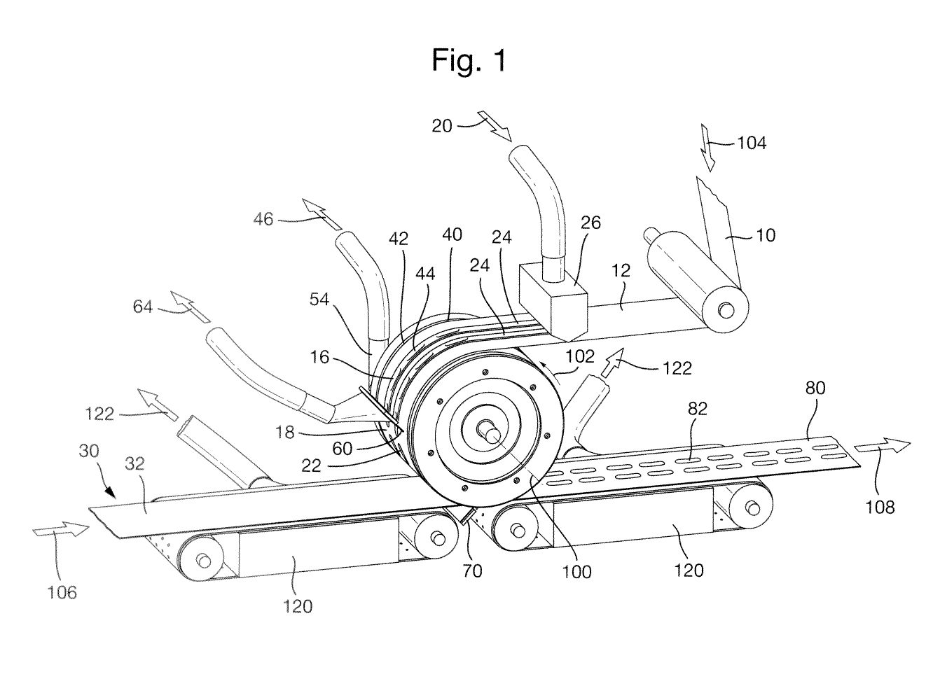

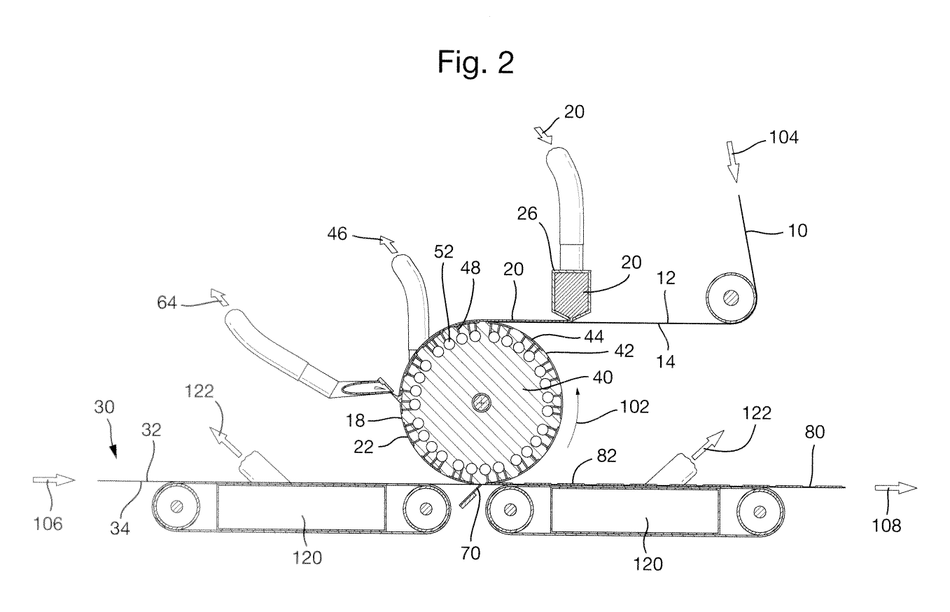

Referring to FIGS. 1 and 2, substrate 10 is supplied to the process in the direction of arrow 104. The substrate 10 may consist of a continuous web or discontinuous material including discrete sheets. The personal care composition 20 is applied to the first surface 12 of the substrate 10 using an applicator 26. The applicator 26 may comprise a slot coater, delivery through a nozzle or multiple nozzles, delivery through an aperture or multiple apertures, flood coating by submerging the substrate 10 in a bath, spraying of atomized personal care composition particles, or other means of application known to one skilled in the art. The mass flow rate of personal care composition 20 addition is preferably greater than or equal to the resulting product containing discrete areas of coated personal care composition 22.

Preferably the personal care personal care composition 20 is applied to the first surface 12 of the substrate 10 before the substrate is brought into contact with the support member 40. While the personal care composition 20 can be applied to the substrate 10 while in contact with support member 40, the applicator 26 would then need to apply personal care composition 20 against a non-flat surface due to substrate 10 conforming to the cavities 44 in the support member 40. For an applicator 26 such as a slot coater, the irregular surface can lead to discontinuous coating and air entrainment with the personal care composition 20. Applying the personal care composition 20 to the substrate 10 prior to the substrate being brought into contact with the support member 40 provides a flat continuous surface for application. Application can either be against an unsupported tensioned web span of substrate 10 or the substrate 10 can be backed by a smooth back up roller or dead plate.

Preferably the personal care composition 20 will be applied in a continuous stripe 24 corresponding to the cross machine direction position of cavities 44 on support member 40. This will minimize the distance that the accumulated personal care composition 68 will need to travel across the width of the wiping member 60 resulting in less excess addition of personal care composition. Alternatively the stripe 24 could be discontinuous and pulsed in time to correspond with the sequential machine direction position of cavities 44 on support member 40.

A support member 40 is provided with a support surface 42 wherein the second surface 14 of the substrate 10 is in contact with the support surface 42 of the support member 40, the support surface 42 having at least one cavity 44 corresponding to the treatment area 16. The support member 40 may be a flat plate or other ruled surface capable of supporting the substrate 10 during coating. Alternatively it may be rotatable. In a preferred embodiment, the support member 40 is a rotatable cylinder with cavities 44 distributed on the surface. The rotatable cylindrical support member 40 can enable a continuous coating process which is capable of high speeds. Alternatively, a cylindrical support member 40 could also be employed in either an indexing, or other non-constant speed process. The preferred cylindrical support member 40 illustrated in FIGS. 1-3 rotates about axis 100 in direction 102. Preferably the support surface 42 will have a very smooth or polished finish with an roughness average of less than approximately 1.6 micrometers. The support surface 42 is also preferably a rule surface. In the case of a cylindrical support member 40 the support surface preferably should not deviate more than approximately 0.1 mm from a true cylinder except for the regions defined by cavity 44.

The final shape of any discrete area of personal care composition 22 once deposited on the substrate 10 is determined by the shape of the cavity 44 of the support member 40. The profile and size of the cavity 44 can be varied to deliver the desired shape of personal care composition 22. The spacing between multiple cavities corresponds to the desired spacing between coated areas of personal care composition 22. The process is capable of producing large shapes of personal care composition 22 with features measuring at least 1000 times the coating thickness. The smallest feature that can be resolved is limited to roughly 0.5 to 5 times the coating thickness. In addition to creating discrete areas of personal care composition 22, this process may also be employed to create a continuous pattern of personal care composition. By varying the depth of the cavity 44 in the support surface 42, the thickness of the personal care composition upon the substrate can be determined. For example if the depth of the cavity 44 is 0.25 mm below the support surface 42, than the resulting thickness of the discrete area of coated personal care composition 22 will be approximately 0.25 mm. Reducing the depth of the cavity 44 will result in a reduced thickness of coated personal care composition 22. Preferably the depth of the cavity 44 is such that the personal care composition is disposed on the substrate in an amount per unit area of the coated region of from 0.300 g/cm² to 0.001 g/cm², more preferably from 0.015 g/cm² to 0.003 g/cm², even more preferably from 0.080 g/cm² to 0.005 g/cm² and even more preferably still from 0.05 g/cm² to 0.005 g/cm².

A cavity 44 with uniform depth relative to the support surface 42 will result in a personal care composition coating 22 of substantially uniform depth. A cavity 44 of varying depth relative to the support surface 42 will result in a personal care composition coating 22 of varying depth. This can be used to create a personal care composition coating 22 targeted to provide more personal care composition benefits to some areas and less to other areas.

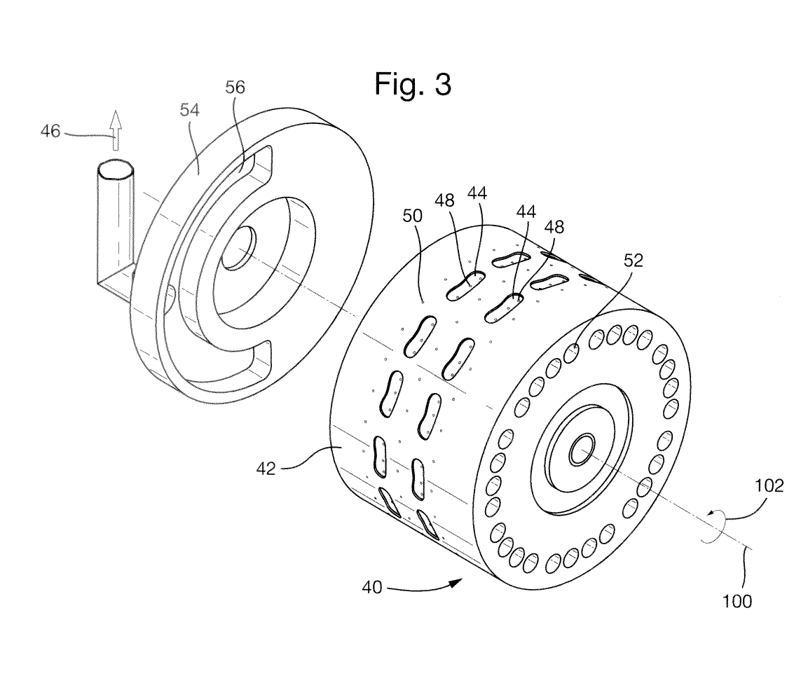

Friction between the second surface 14 of the substrate 10 and the support surface 42 supports and controls the substrate 10 so that the treatment areas 16 of the substrate 10 can deform and conform to the shape of the cavity 44 during coating with minimal distortion to the remainder of the substrate 10 outside of the treatment area 16. The resulting stress created due to the deformation of the substrate 10 into cavity 44 is less than the elastic limit of the substrate 10, and as such the final coated substrate 10 will not permanently deform. The elastic limit is measured as defined in ASTM D 638-02; in particular, in section A2.1. The resulting product will be a flat substrate 10 with discrete areas of coated personal care composition 22 above the flat first surface 12 of substrate 10. Friction between the second surface 14 of the substrate 10 and the support surface 42 of a cylindrical support member 40 also provides traction to drive the substrate 10. Referring to FIG. 3, friction between the second surface 14 of the substrate 10 and the support surface 42 of the support member 40 can be increased by applying a negative pressure 46 or vacuum source to substrate control vacuum holes 50. Sufficient negative pressure 46 applied to substrate control vacuum holes 50 can lock the substrate 10 against the support surface 42 during coating.

Referring to FIGS. 1,2, 4, and 6; in a preferred embodiment, a wiping member 60 is positioned along the path of the substrate 10 coated with a stripe of personal care composition 24 while the second surface of the substrate 14 is in contact with support member 40. The wiping member 60 is therefore in contact with the first surface of the substrate 12 and the stripe of personal care composition 24. Preferably the wiping member is cantilevered and supported at a first end by a rigid holder. A second end is in contact with the first surface of the substrate. The wiping member is preloaded against the substrate by the support to apply and maintain force against the substrate. This force allows the wiping member to clean residual gel from the substrate. The wiping member has some flexibility to bend in the machine direction to accommodate changes in substrate thickness and variation in radius of the support member and maintain the force applied to the substrate.

There is relative motion between the wiping member 60 and the support member 40 with substrate and personal care composition. Preferably the support member 40 will move and the wiping member 60 is stationary. Alternatively it is also possible to configure the process such that the wiping member 60 is moving and the support member 40 is stationary or moving at a different velocity than the wiping member. The wiping member 60 distributes the personal care composition stripe 24 to fill the volume of the cavity 44 lined with substrate 10. Accumulated excess personal care composition 68 accumulates in front of the wiping member 60. Hydraulic pressure is generated in the accumulated personal care composition 68 by the relative motion of the stripe of personal care composition 24 carried by the substrate 10 and the wiping member 60. The hydraulic pressure generated can deform the substrate 10 and cause it to conform to the cavity 44. Preferably negative pressure 46 will also be applied to the cavity 44 to ensure the substrate 10 fully conforms to the volume of the cavity 44 in the treatment area 16. The substrate 10 conforms to the shape and depth of the cavity 44. The substrate 10 is coated by the personal care composition 20 in the area corresponding to the shape and depth of cavity 44. The substrate 10 is not permanently deformed during this process which will allow the substrate to remain flat when removed from the cavity 44.

The wiping member 60 removes some or preferably substantially all, of the personal care composition 20 from the first surface of the substrate 12 in the areas outside the treatment area 16. This results in a clean substrate 18 with discrete coated areas of personal composition 22. Some or all of the personal care composition 68 removed from the clean area of the substrate 18 may be accumulated and redistributed to fill successive coated areas 22. Excess personal care composition 66 not needed for coating can be excised to be recycled or disposed of. Preferably, the excised personal care composition 66 is removed from the wiping member by vacuum 64. Adjusting the pick up location and geometry of the vacuum nozzle can adjust the amount of accumulated personal care composition 68. Flow of excised personal care composition 66 conveyed by vacuum can be assisted by solvent impingement which prevents accumulation of personal care composition on the vacuum ducting. Excised personal care composition 66 can also be removed by pumping or by distributing into lanes on the substrate 10 in regions that will not be used for the final product.

The wiping member 60 preferably at least partially consists of a metal or plastic blade because a blade has a shape advantageous to removing the maximum amount of composition from the first surface of the substrate. Alternatively the wiping member 60 could be a roller covered in elastomeric material, a brush, or a plurality of flexible fingers, etc. The wiping member 60 is preferably able to conform to irregularities and changes in the radius of support surface 42 and comprises and edge, where said edge provides a removal force. This further allows the wiping member 60 to remove excess personal care composition and creates a clean surface 18. The wiping member 60 is preferably stiff across its width to prevent the wiping member from conforming to the cavities 44 resulting in a generally uniform thickness for the discrete area of personal care composition 22 for a cavity 44 of constant depth. If the wiping member 60 were not stiff across its width and able to conform to the cavities 44, the resulting discrete area of personal care composition 22 would have significantly lower depth of personal care composition in the center due to removal of personal care composition by the bending wiping member 60.

Some examples of suitable construction for wiping member 60 are stainless steel, brass, titanium, or other metal approximately 0.05-2.0 mm thick; ABS, Delrin, PTFE, fiber glass, carbon fiber, ceramic, glass or other plastic or composite material approximately 0.05-4.0 mm thick; or polyurethane, rubber, or other elastomeric material approximately 0.05-10.0 mm thick preferably backed up by another stiffer material to provide stiffness across the width. Preferably the edge of wiping member 60 in contact with the second surface of substrate 12 will be ground to a knife edge. Such a tapered knife edge allows some flexibility to conform to small imperfections yet provides stiffness across the width. Alternatively the edge of wiping member 60 in contact with the second surface of substrate 12 can have a radiused, filleted, chamfered, or square edge. Preferably the edge in contact with the second surface of substrate 12 will have a very smooth or polished finish with and roughness average of less than about 1.6 micrometers. Alternatively the edge of wiping member 60 in contact with the second surface of substrate 12 can be wrapped in a conformable tape such as PTFE or polyurethane.

The wiping member 60 forms a contact angle 62 with a tangent line constructed at the contact point of the wiping member 60 and the first side of the substrate 12 and the surface of the first side of substrate 12 as it conforms to support surface 42. Preferably the contact angle 62 is from 1° to 179°, preferably from 10° to 170°, preferably from 10° to 70°, more preferably from 20° to 55°, even more preferably from 30° to 45° and even more preferably still from 35° to 40°. Adjustment of the contact angle 62 impacts how effectively the wiping member 60 will substantially clean substrate 18.

Referring to FIG. 3, preferably negative pressure 46 is applied to a cavity vacuum hole 48 coincident with the cavity 44 resulting in the substrate 10 being drawn into the cavity 44. Referring to FIGS. 1, 2, and 3, for a cylindrical support member 40, negative pressure 46 to supply substrate control vacuum holes 50 and cavity vacuum holes 48 is preferred while the second surface 12 of substrate 10 is in contact with support surface 42. The timing of negative pressure 46 is controlled by a stationary vacuum manifold 54. The vacuum manifold 54 contains an arcuate passage 56 that provides continuity of negative pressure to vacuum supply ports 52. Arcuate passage 56 distributes negative pressure 46 from the vacuum manifold 54 to the support member 40 depending on the rotational position of the supply port 52 relative to the arcuate passage 56. Vacuum holes 48 and 50 are supplied by vacuum supply port 52. Adjusting the endpoint positions of the arcuate passage 56 adjusts the timing for when negative pressure will turn on and off for vacuum holes 48 and 50. Independent timing and vacuum level for cavity vacuum holes 48 and substrate control vacuum holes 50 could be achieved by providing a second concentric arcuate passage on manifold 54 that supplies a second concentric set of vacuum supply ports 52. Optionally a positive pressure can be supplied at the end of the arcuate passage 56 to help separate the coated substrate 10 from support member 40. Alternative independent vacuum supply pressure and timing could be supplied for each side or a number of other approached familiar to one skilled in the art.

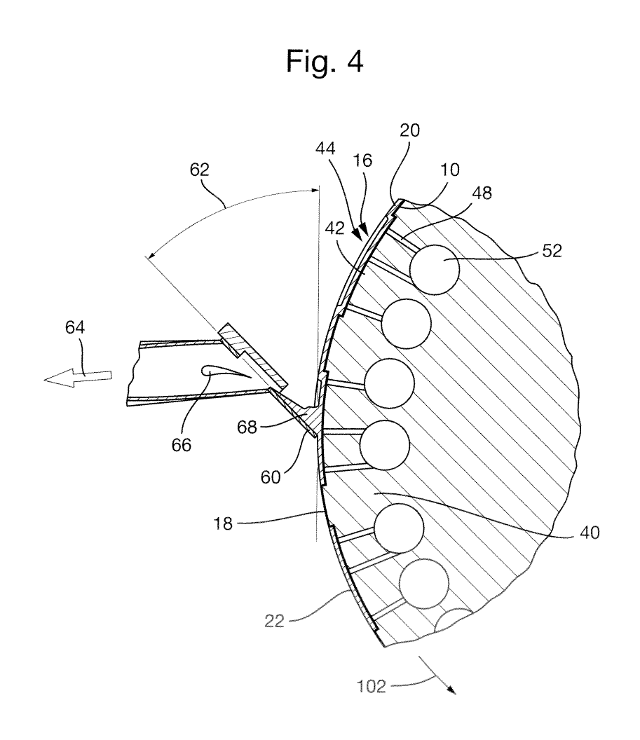

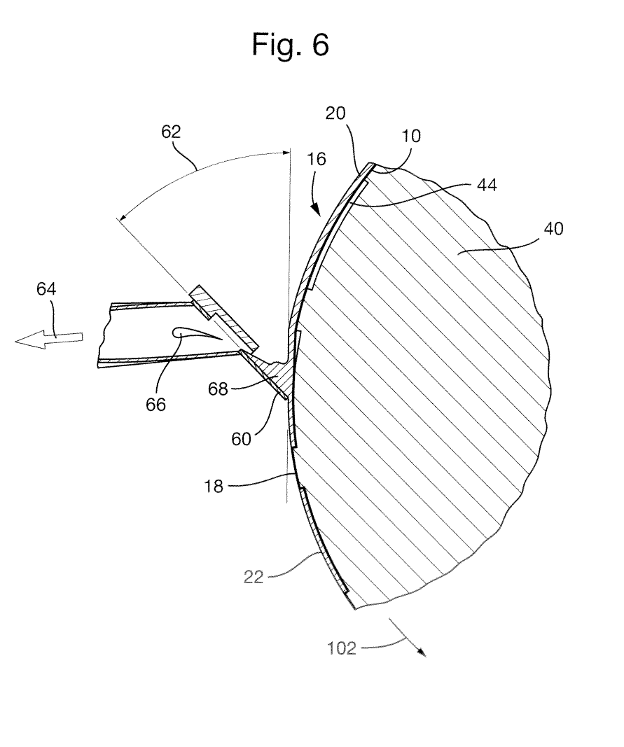

Preferably negative pressure 46 is applied to vacuum holes 48 to pull the substrate 10 stripe of personal care composition 24 into the cavities 44 before approaching the wiping member 60. FIG. 4 shows a cross-section of the support member 40 where negative pressure 46 is applied to the cavities 44. Alternatively it is possible to coat discrete areas of personal care composition 22 without applying negative pressure 46 to the cavities 44 before the wiping member 60 or no negative pressure applied at all. FIG. 6 shows a cross section of the support member 40 where negative pressure 46 is not applied to the cavities 44.

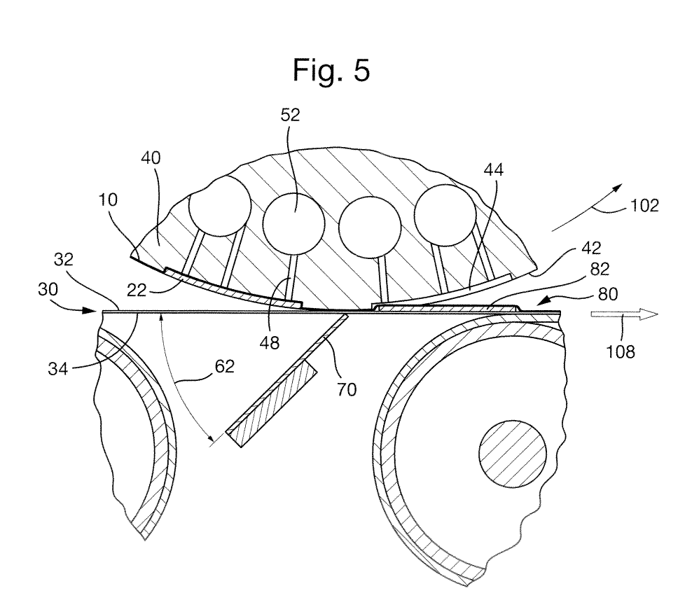

Referring to FIGS. 1, 2, 4, and 5; after the support member 44 with the substrate 10 moves past the wiping member 60, the resultant substrate has discrete areas of personal care composition 22 surrounded by areas of clean substrate 18. The discrete areas of personal care composition 22 continue to fill the cavities 44 as long as negative pressure is applied to cavity vacuum holes 48.

It may be desirable to combine the substrate 10 coated with a discrete area of personal care composition 22 with a second substrate 30 which may act as a release liner in order to protect the coated substrate. The second substrate can be supplied to the process in the direction of arrow 106. The second substrate 30 may comprise a continuous web or discontinuous material including discrete sheets. A composite structure can be created where the first surface 12 of substrate 10 faces the first surface 32 of the second substrate 30. The discrete area of personal care composition would then be sandwiched between substrate 10 and the second substrate 30. The combination of the second substrate 30 is optional and the herein described process can be used to solely coat discrete areas of personal care composition 22 onto a single substrate 10.

Preferably, combining of the second substrate 30 will occur while the substrate 10 remains in contact with the support member 40. This provides the advantage of preventing compression and spreading of the personal care composition between the layers of substrate 10 and second substrate 30 because the discrete area of personal care composition 22 is protected by the cavity 44 because it can occupy the volume of the cavity 44 during combining. Preferably the second substrate 30 will be fed tangentially with the support member 44. Optionally, a second wiping member 70 can be used to encourage intimate combination of the substrate 10 and second substrate 30. This can exclude air and oxygen that would otherwise reside between the substrates. The second wiping member 70 can be of similar construction of the wiping member 60 described herein. The second wiping member 70 is adjusted to form a contact angle 62 in a similar range of adjustment as for wiping member 60.

The preferred combined coated composite web 80 consists of substrate 10 and second substrate 30 with discrete areas of coated personal care composition 82 between. Combined coated composite web is fed away from the process in the direction of arrow 108.

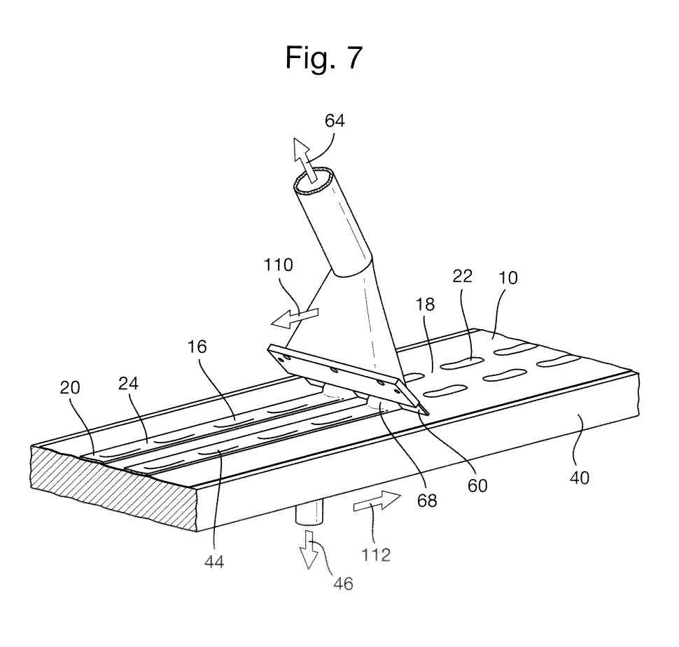

Preferably the support member 40 will be in the shape of a cylinder as illustrated in FIGS. 1-6. An alternative embodiment uses a non-cylindrical support member 40. This support surface 42 could be flat or curved. FIGS. 7-9 illustrate an alternative embodiment wherein the support surface 42 is flat. The flat support surface 42 can comprise continuous or discontinuous surfaces. The support member 40 could be executed as discrete rigid flat plates, discrete rigid platens connected together by a chain or belt, flexible belt material, etc. The flat support surface 42 lends itself to an intermittent indexing motion, however continuous motion is also possible with an endless belt or chain or using a walking beam mechanism. As with the preferred rotary embodiment the flat support member 40 will include cavities 44 corresponding to the treatment area 16.

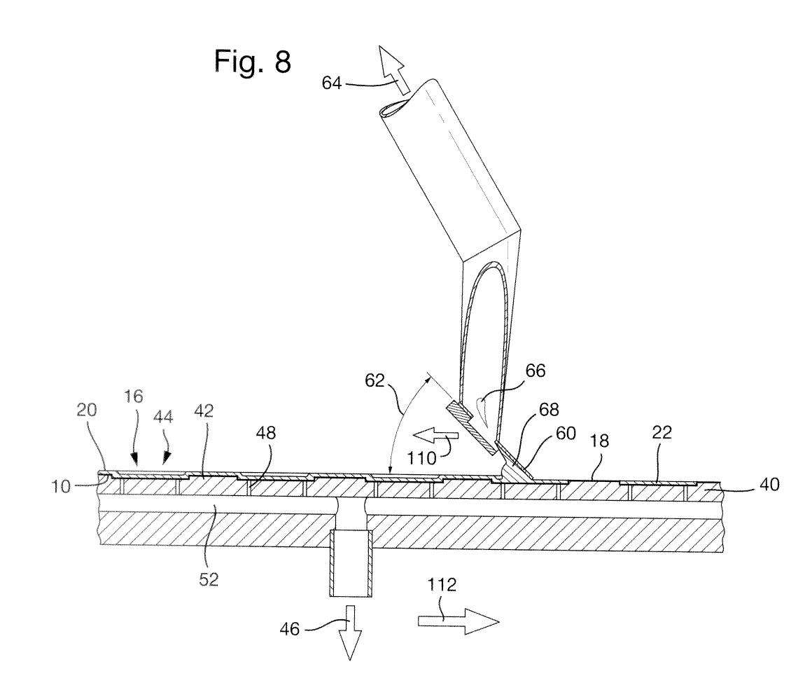

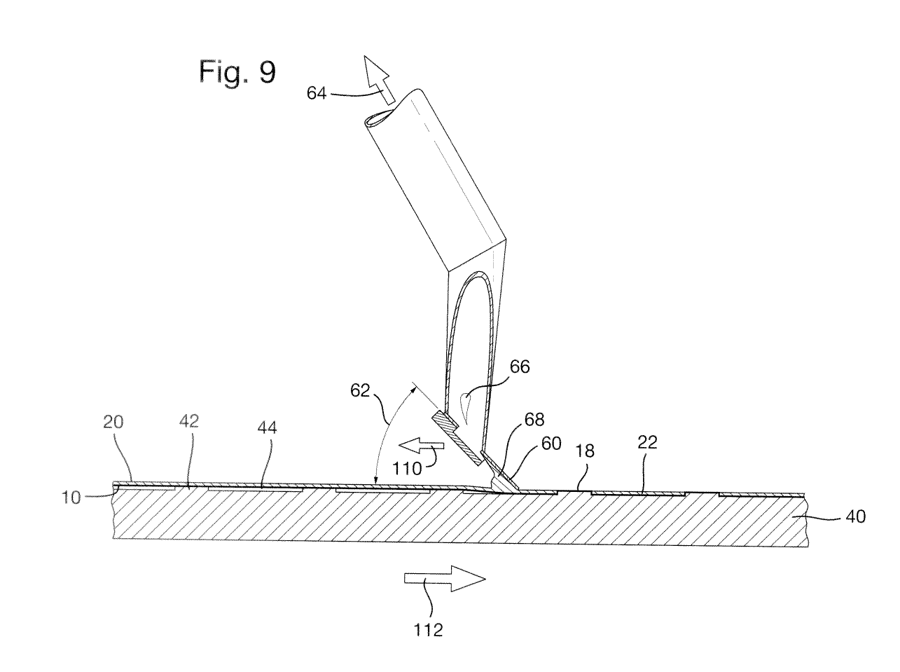

The process with a flat support member 40 is the same as with a cylindrical rotary support member 40. Preferably stripes of personal care composition 24 are applied to substrate 10. Substrate 10 is fed or placed onto the support surface 42. Relative linear motion between the flat support member 40 and wiping member 60 forces personal care composition 20 and substrate 10 into cavities 44 and removes excess personal care composition to clean substrate 18. Direction of motion for support member 40 is indicated by arrow 112 and arrow 110 indicates direction of linear motion for wiping member 60. Both support member 40 and wiping member 60 can be moving or one can be stationary. As with the rotary embodiment, preferably the cavities 44 will have negative pressure 46 supplied by vacuum holes 48. FIG. 8 illustrates a cross-section of the support member 40 where negative pressure 46 has been applied to the substrate 10 to draw it into the cavities 44. Alternatively the process can be executed without the addition of negative pressure, relying on the hydraulic pressure generated by the relative motion between the wiping member 60 and the stripes of personal care composition 24 to press the substrate and personal care composition 22 into the cavities 44. FIG. 9 illustrates a cross-section of the support member 40 where negative pressure 46 is not applied. After the wiping member passes by, the resulting substrate 10 will have discrete areas of personal care composition 22 surrounded by clean areas 18. Alternatively a second substrate can be combined while the substrate 10 is in contact with the flat support surface 42 or the coated substrate 10 can be removed from the support surface 42.

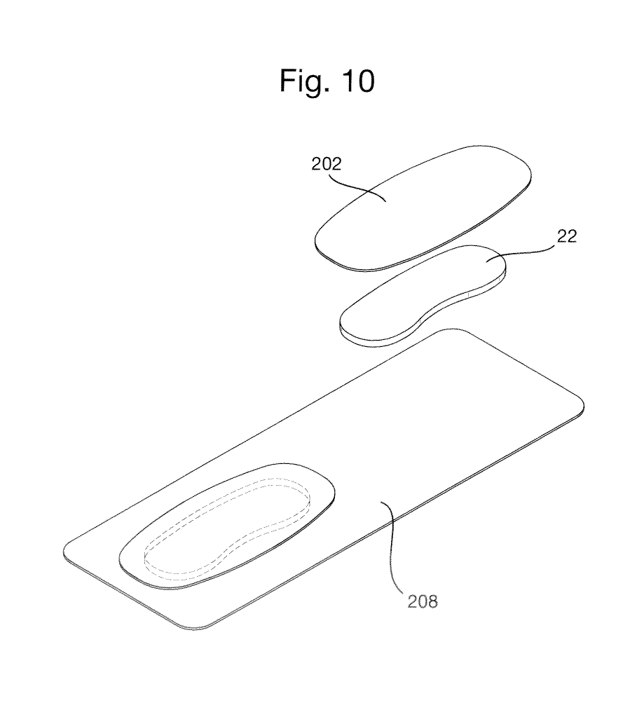

FIG. 10 illustrates a partially exploded view of a preferred embodiment for the final personal care article for use by the end consumer. The discrete area of personal care composition 22 is created by the herein described process. The substrate 10 is cut into a shape 202 which will allow the consumer to apply the personal care composition 22. The second substrate 30 is cut into a shape 208 to form a card to carry one or more substrate 202 and personal care composition 22. The personal care composition 22 and substrate 202 are preferably different shapes with the substrate area 202 larger than the personal care composition 22. This will provide an area for the consumer to hold the substrate that is not coated in personal care composition. The shape of the second substrate 208 is preferably different and larger than the shape of the substrate 202. The shapes in FIG. 10 are purely for illustrative purposes. Many other shapes are possible.

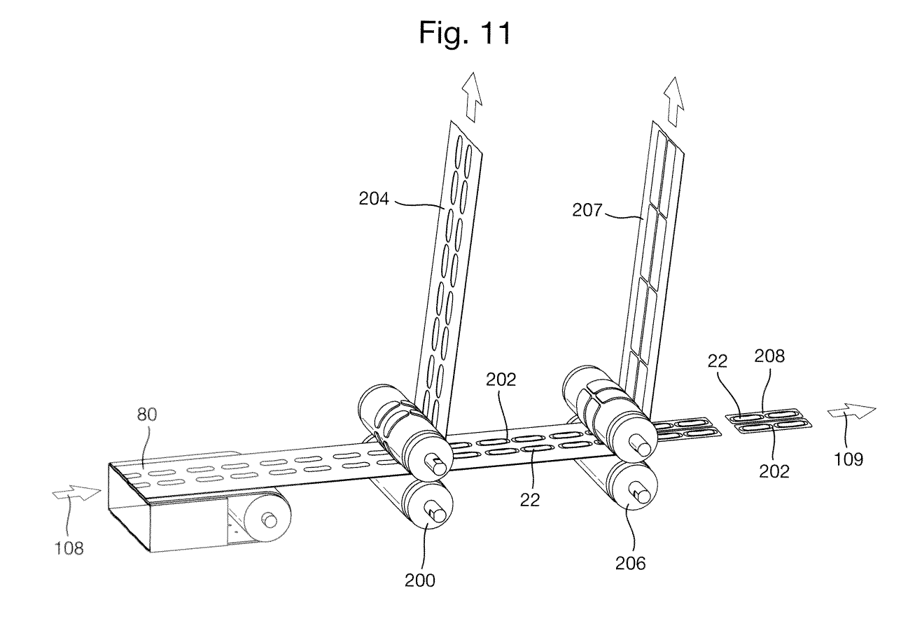

Referring to FIG. 11, the coated composite web 80 travels in direction of arrow 108. Knife 200 can be used to cut the shape 202 in the substrate 10. The knife 200 is preferably a kiss cut knife that will cut through the substrate 10 but not through second substrate 30 in order to cute the first substrate into an area that is larger and unique from the area coated by the personal care composition, without severing the second substrate. The knife 200 is preferably a rotary die cutter with sufficient clearance between the blade tip and anvil to enable the kiss cut of substrate 202 only. Trim material 204 leftover from cutting shape 202 can be pulled away from the process for recycling or disposal.

A second knife 206 can be used to cut shape 208 into second substrate 30 such that the second substrate is larger than the cut area in the first substrate. Knife 206 is preferably a rotary die cutter set up to cut completely through second substrate 30. Trim material 207 leftover from cutting shape 208 can be pulled away from the process for recycling or disposal. Final product cards 208 may be transported away by conveyor or other means in the direction of arrow 109. The final product cards 208 can next be packaged into secondary package such as a pouch, bag, tray, or carton. Preferably the cards 208 will be packed into a foil pouch after purging all oxygen inside of the pouch with an inert gas or Nitrogen. This will protect personal care composition 22 from degradation by exposure to Oxygen.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for coating a personal care composition onto at least one discrete treatment area of a substrate having a first and second surface wherein the personal care composition has a yield point of from 10 Pa to 2000 Pa measured via a stress controlled amplitude sweep at a frequency of 1 Hz and a temperature of 25° C. comprising the steps of:
a. applying the personal care composition to a portion of the first surface of the substrate
b. providing a support member with a support surface wherein the second surface of the substrate contacts the support surface of the support member, the support surface having at least one cavity corresponding to the treatment area;
c. providing a wiping member in contact with the first surface of the substrate;
d. deforming the substrate with the applied personal care composition on its first surface into the cavity such that the resulting stress created due to the deformation of the substrate into cavity is less than the elastic limit of the substrate;
e. providing relative motion between the support member and the wiping member such that excess personal care composition is removed and the substrate is coated with a discrete area of personal care composition substantially corresponding to the cavity; wherein the substrate is not permanently deformed; and
f. removing the substrate from the support member.

2. A process according to any preceding claim wherein the personal care composition is a depilatory composition.

3. A process according to any preceding claim wherein the support member is rotatable, preferably wherein the support member is cylindrical.

4. A process according to any preceding claim wherein the support member is coincidental with a negative pressure applied to the cavity.

5. A process according to any preceding claim, wherein excess personal care composition is at least partially removed from substrate, preferably by means of scraping, scrubbing, suction, solvent impingement, solvent immersion and mixtures or combinations thereof, more preferably by means of scraping by the wiping member, even more preferably by means of scraping by the wiping member and by suction, such that the substrate is substantially free of personal care composition with the exception of the treatment area.

6. A process according to any preceding claim wherein a first surface of a second substrate is placed in contact with the coated substrate before the coated substrate is removed from the support member.

7. A process according to any preceding claim wherein the wiping member comprises a blade.

8. A process according to any preceding claim wherein the wiping member forms a contact angle of from 1° to 179°, preferably from 10° to 170°, preferably from 10° to 70°, more preferably from 20° to 55°, even more preferably from 30° to 45° and even more preferably still from 35° to 40°.

9. A process according to any preceding claim wherein excess personal care composition is removed from the wiping member by means of a vacuum.

10. A process according to any preceding claim wherein the personal care composition is applied to the first surface of the substrate in a continuous stripe before the substrate is brought into contact with the support member.

11. A process according to any preceding claim, wherein the personal care composition is disposed on the substrate in an amount per unit area of the coated region of from 0.300 g/cm² to 0.001 g/cm², more preferably from 0.015 g/cm² to 0.003 g/cm², even more preferably from 0.080 g/cm² to 0.005 g/cm² and even more preferably still from 0.05 g/cm² to 0.005 g/cm².

12. A process according to any preceding claim, wherein the substrate has a rigidity in the range of from 5.00 g/cm to 0.08 g/cm, preferably from 3.00 g/cm to 0.08 g/cm, more preferably from 1.80 g/cm to 0.10 g/cm, even more preferably from 0.80 g/cm to 0.15 g/cm and even more preferably still from 0.60 g/cm to 0.25 g/cm.

13. A process according to any preceding claim wherein the substrate is water impermeable, preferably wherein the substrate comprises a water impermeable material, more preferably a plastic sheet, even more preferably a polyolefin, even more preferably still a polyethylene and yet more preferably high density polyethylene.

14. A process according to any preceding claim wherein the process further comprises a knife which cuts the first substrate in an area that is larger and unique from the area coated by personal care composition without severing the second substrate and wherein a second knife cuts the second substrate in an area that is larger than the cut area in the first substrate.

15. A process according to any preceding claim wherein the cavity in the support surface includes areas of non-uniform depth.
